(19) United States
(12) Patent Application Publication
(10) Pub. No.: US 2009/0293138 A1
(43) Pub. Date: Nov. 26, 2009

(54) ANIMAL MODELS CARRYING TUMORS EXPRESSING HUMAN PROSTATE CANCER-SPECIFIC ANTIGEN AND METHOD FOR ANALYZING PREVENTION AND TREATMENT EFFICACY OF DENDRITIC CELLS-DERIVED IMMUNOTHERAPEUTICS

(75) Inventors: Dong Sung Lee, Daejeon (KR); Woo Bok Lee, Gyeonggi-do (KR); Choon Ju Jeon, Suwon Si (KR); Yong Soo Bae, Gyeonggi-do (KR)

Correspondence Address: THE RAFFERTY PATENT LAW FIRM, 1952 Gallows Road, Suite 200, Vienna, VA, 22182-3823, US

(73) Assignee: CREAGENE, INC., Seoul (KR)

(21) Appl. No.: 11/915,737

(22) PCT Filed: May 27, 2006
(86) PCT No.: PCT/KR2006/002028

§ 371 Date: Feb. 13, 2008

(30) Foreign Application Priority Data
May 27, 2005 (KR) 10-2005-0045236

(51) Int. Cl. A01K 67/033; A61K 49/00; C12N 5/06; A61P 35/00

(52) U.S. Cl. 800/10; 424/9.2; 435/354

(57) ABSTRACT

A method for analyzing the prevention and treatment efficacy of a dendritic cell-derived immunotherapeutic for prostate cancer using an animal model carrying tumors expressing a human prostate cancer-specific antigen includes either administering to a normal non-human animal dendritic cells to be analyzed, or administering to a normal non-human animal a cancer cell line expressing the human prostate cancer-specific antigen to induce cancer in the normal non-human animal; administering to the animal the cancer cell line expressing the human prostate cancer-specific antigen to induce cancer in the animal when the dendritic cell administering step was performed, or administering to the animal with cancer dendritic cells to be analyzed when the human prostate cancer-specific antigen expressing cell line administering step was performed; and determining the prevention and treatment efficacy of the dendritic cells as immunotherapeutics for prostate cancer by measuring the formation or growth of cancer cells originated from the cancer cell line in the animal.

### CROSS-REFERENCES TO RELATED APPLICATIONS

This is a US National Stage of International Application PCT/KR2006/002028, filed Nov. 27, 2006, which was published as International Publication WO 2006/126864 on Nov. 30, 2006. The benefit of priority is further claimed to Republic of Korea patent application KR 10-2005-0045236, filed May 27, 2005, which was registered as Republic of Korea Patent Reg. No. 10-0647847 on Nov. 23, 2006. Each application to which the benefit of priority is claimed is incorporated herein by reference in its entirety.

### INCORPORATION OF SEQUENCE LISTINGS

The accompanying Sequence Listings, including SEQ ID NO:1 through SEQ ID NO:10, which were submitted electronically in computer-readable format in the file having filename “P10072-SEQ1.txt,” 22 kB in size, are incorporated herein by reference in their entireties.

### FIELD OF THE INVENTION

The present invention relates to a method for analyzing the prevention and treatment efficacy of a dendritic cell-derived immunotherapeutic for prostate cancer, more particularly, to a method for a method for analyzing the prevention and treatment efficacy of a dendritic cell-derived immunotherapeutic for prostate cancer using an animal model bearing human prostate cancer.

### BACKGROUND

The prostate gland is a male organ that produces a component of seminal fluid, and which is located under the bladder and adjacent to the rectum. Prostate cancer is mostly caused by the canceration of gland cells in the prostate gland and is very likely to metastasize to the lymph nodes and bones. 90% of prostate cancers are proliferated with the help of male sex hormones generated in the body. Therefore, it is a prevalent therapy for prostate cancer to prevent the proliferation of cancer and to lyse cancer cells by inhibiting the action of male sex hormones (Greenlee, R. T et al., Cancer statistics. CA Cancer J. Clin. 50:7 (2000)).

Recently, the incidence of prostate cancer has increased with the highest rate in Korea and is found mostly in men over fifty years of age. Prostate cancer, occupying about 20% of male cancers, is the most prevalent cancer in the USA and Europe. In the USA, prostate cancer shows the highest incidence among male cancers and the second highest mortality behind lung cancer. The number of people in Korea suffering from prostate cancer has increased due to the increase in life span among men and the westernization of food. Such increasing patterns have become more prominent in the last decade. According to records of cancer patients in Korea, the proportion of prostate cancer patients among male cancers is 2.7% and 3.0% in the year of 2001 and 2002, respectively. It could be anticipated that the incidence of prostate cancer will further increase rapidly.

The therapy for prostate cancer includes (a) radical prostatectomy, (b) radiotherapy, (c) chemotherapy and (d) hormone therapy. The most prevailing therapy is a hormone therapy in which the secretion of a male sex hormone is inhibited or its production is blocked by androgen removal. The hormone therapy exhibits temporary treatment effects in more than 80% of patients such as the inhibition of cancer growth and reduction of lesion. However, in most of patients showing favorable responses to the temporary hormone therapy, hormone-refractory prostate cancer (HRPC) is very likely to be developed. Patients having HRPC are died within one year from diagnosis. HRPC are hardly treated with conventional anticancer drugs, chemotherapy and radiotherapy, so that there remains a need for a novel therapy for prostate cancer (Fong, L. et al., Induction of tissue-specific autoimmune prostatitis with prostatic acid phosphatase immunization: implications for immunotherapy of prostate cancer. J. Immunol. 159:3113. (1997)).

Recently, as the immunotherapy using cytokines and dendritic cells becomes promising, dendritic cell-based therapeutics (DC vaccines) have been suggested as a novel immunotherapy for HRPC ((Fong, L. et al., Dendritic cells in cancer immunotherapy. Annu. Rev. Immunol. 18:245. (2000); Xue B H. et al., Induction of human cytotoxic T lymphocytes specific for prostate-specific antigen. Prostate. 30:73.78. (1997)). For clinical tests of immunotherapy using dendritic cells, it is prerequisite to examine their efficacy and safety in animal models. However, there has not been yet proposed prostate cancer animal models for evaluating dendritic cell-based vaccines against human prostate cancer.

Throughout this application, several patents and publications are referenced and citations are provided in parentheses. The disclosure of these patents and publications is incorporated into this application in order to more fully describe this invention and the state of the art to which this invention pertains.

### SUMMARY

Endeavoring to meet the need in the art described above, the present inventors have established xenogenic cancer cell lines expressing human prostate cancer-specific antigens and animal models using them. In addition, we have found that the prevention and treatment efficacy of dendritic cells as immunotherapeutics for prostate cancer could been accurately analyzed using the animal models.

Accordingly, it is an object of this invention to provide a method for analyzing the prevention and treatment efficacy of a dendritic cell-derived immunotherapeutic for prostate cancer.

It is another object of this invention to provide a mouse-derived renal cancer cell line expressing a human prostate cancer-specific antigen.

It is still another object to this invention to provide a mouse (Mus musculus) prostate cancer model.

Other objects and advantages of the present invention will become apparent from the detailed description to follow taken in conjugation with the appended claims and drawings.

### BRIEF DESCRIPTION OF THE DRAWINGS

FIG. 1 is a gel photograph showing PCR products of nucleotide sequences encoding prostate-specific antigens PCA, PSCA, PSA, PAP and PSMA. For preparing recombinant antigens, cDNA molecules were synthesized from LNCaP.FGC (human prostate cancer cell line) and used for PCR amplification of nucleotide sequences encoding prostate-specific antigens PCA, PSCA, PSA, PAP and PSMA. Each PCR amplicon represents a full length sequence lack of signal sequence and transmembrane domain. Lanes M, 1, 2, 3, 4 and 5 denote 1 kb ladder marker, PAP (amino acids 33-386; 1.062 kb), PSA (amino acids 19-261; 0.729 kb), PSMA (amino acids 394-707; 0.94 kb), PCA (amino acids 30-204; 0.52 kb) and PSCA (amino acids 23-93; 0.21 kb), respectively.

FIG. 2 represents genetic maps and their partial nucleotide sequences of recombinant vectors for expressing prostate cancer-specific antigens. Using cDNA molecules synthesized from LNCaP.FGC (human prostate cancer cell line) as templates, nucleotide sequences encoding prostate-specific antigens PCA, PSCA, PSA, PAP and PSMA were amplified by PCR. For expressions, the amplified sequences were cloned into either a eukaryotic vector (panel A) or prokaryotic vector (panel B). In the genetic map of vectors, HA, 36A, P_{CMV}, BGH pA, f1 ori and SV40 ori represent hemagglutinin-encoding sequence, 36A Tag-encoding sequence, promoter of cytomegalovirus, polyadenylation sequence of bovine growth hormone gene, f1 replication origin and SV40 replication origin, respectively. The antibiotics represent antibiotic-resistant genes.

FIG. 3 shows the results of Western blotting for prostate cancer antigens expressed in transformed cells. RENCA cells were transformed with the recombinant pcDNA3.1-HA-36A/PAP, pcDNA3.1-HA-36A/PSA or pcDNA3.1-HA-36A/PCA and selected in the presence of antibiotics G418, followed by Western blotting. As an antibody for analysis, 36A Tag-specific monoclonal antibody was used. Lanes M, 1, 2, 3 and 4 represent the results of a molecular weight marker, RENCA, RENCA/PAP, RENCA/PSA and RENCA/PCA, respectively.

FIG. 4 represents the results of Western blotting showing the expression stability of prostate cancer antigens (PAP, PSA and PCA) introduced into RENCA cell. Cell lines established were cultured in the absence of G418 and 1×10⁶ cells were subjected to Western blotting. Nc denotes a negative control, non-transformed RENCA cells.

FIG. 5 shows the results of SDS-PAGE analysis and Western blotting analysis for prostate cancer antigens (PCA, PSCA, PAP, PSA and PSMA). The nucleotide sequences encoding prostate cancer antigens were cloned into pCTP vector and expressed in BL21-gold(DE3). The recombinant CTP-conjugated proteins expressed were confirmed by 12% SDS-PAGE and Western blotting. Lanes M, 1, 2, 3, 4 and 5 correspond to molecular weight marker, CTP-PCA, CTP-PSCA, CTP-PAP, CTP-PSA and CTP-PSMA, respectively.

FIG. 6 shows the results of Western blotting for splenocytes harboring CTP-conjugated prostate cancer antigens. Mouse splenocytes were treated with 50 μg of the CTP-conjugated PCA, PCA, PSCA, PAP, PSA or PSMA. 24-hr later, Western blotting were carried out to verify that the CTP-antigen was introduced into cells. Lanes M and 1-6 correspond to molecular weight marker, a negative control mouse splenocyte, CTP-PCA, CTP-PSCA, CTP-PAP, CTP-PSA and CTP-PSMA.

FIGS. 7a and 7b represent the relative growth rate of solid cancer in BALB/c mice induced by prostate cancer antigen-expressing recombinant RENCA or control RENCA cells. 1×10⁶ cells of recombinant RENCA or control RENCA were subcutaneously inoculated into BALB/c mice and the formation and rate of cancer were observed. Following the inoculation of recombinant tumor cell lines, the size of tumor was measured in a time interval of 2 days.

FIG. 8 represents the prevention effects of DC (dendritic cell)-based vaccines to inhibit tumorigenesis induced by recombinant RENCA cell lines. For investigating the prevention efficacy of DC pulsed with prostate cancer antigens, 1×10⁶ cells/mouse of DC were subcutaneously injected twice into mice in a time interval of one week. 1-week later, 1×10⁶ cells/mouse of recombinant cancer cell lines were subcutaneously injected into mice. Thereafter, the size of tumor was measured in a time interval of 2 days.

FIG. 9 represents the prevention effects of DC-based vaccines to inhibit tumorigenesis. Mice were immunized with DC vaccines and challenged with recombinant cancer cell lines. The formation of cancer was examined in a time interval of 2 days.

FIG. 10 shows the prevention efficacy of DC vaccines to inhibit pulmonary metastasis. Mice were administered twice with DC pulsed with CTP-PAP or CTP-PSA in a time interval of one week. Then, a recombinant prostate cancer cell line (RENCA/PAP or RENCA/PSA) was inoculated into mice via tail vein. After 30 days of inoculation, lung was extracted and photographed, and the number of cancer nodules formed was counted.

FIGS. 11a and 11b represent the treatment efficacy of DC vaccine for cancer in mice harboring tumor. 1×10⁶ cells/mouse of recombinant cancer cell lines expressing human prostate cancer antigens were subcutaneously injected into mice. 3-day later, bone marrow-derived dendtiric cells (Bm-DC) pulsed with a recombinant prostate cancer antigen, CTP-PAP, CTP-PSA or CTP-PCA were subcutaneously injected twice into mice in a time interval of one week. After 2 days of the injection, the formation and size of tumor were examined (FIG. 11a). On 28 day of injection, the tumor in mice was photographed (FIG. 11b).

FIG. 12 represents the activities of cancer antigen-specific cytotoxic T lymphocytes in mice treated with DC vaccines. T lymphocytes were isolated from spleen of mice treated with DC vaccines and mixed with antigen presenting cells (APC) pulsed with each CTP-antigen at a ratio of 5:1 (T:APC). Following 5 days of incubation, the activities of cytotoxic T lymphocytes were measured.

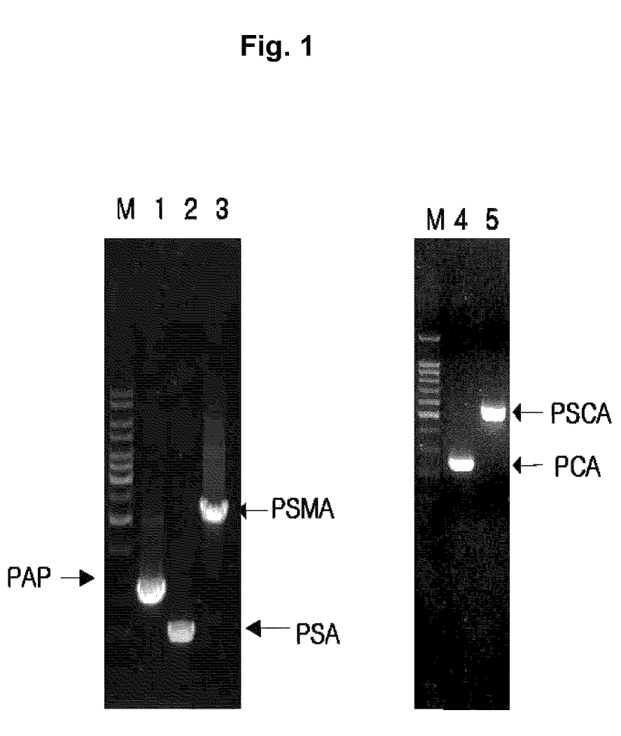

### DETAILED DESCRIPTION

In one aspect of this invention, there is provided a method for analyzing the prevention and treatment efficacy of a dendritic cell-derived immunotherapeutic for prostate cancer using an animal model carrying tumors expressing a human prostate cancer-specific antigen, which comprises the steps of: (a) (a′) administering to a normal animal other than human dendritic cells to be analyzed, or (a″) administering to a normal animal other than human a cancer cell line expressing the human prostate cancer-specific antigen to induce cancer in the normal animal; (b) (b′) administering to the animal the cancer cell line expressing the human prostate cancer-specific antigen to induce cancer in the animal when the step (a′) is performed in the step (a), or (b″) administering to the animal with cancer dendritic cells to be analyzed when the step (a″) is performed in the step (a); and (c) determining the prevention and treatment efficacy of the dendritic cells as immunotherapeutics for prostate cancer by measuring the formation or growth of cancer cells originated from the cancer cell line in the animal.

The present invention is directed to (i) methods for analyzing the prevention efficacy of dendritic cell-derived immunotherapeutic for prostate cancer and (ii) methods for analyzing the treatment efficacy of a dendritic cell-derived immunotherapeutic for prostate cancer.

In this regard, the present method for analyzing the treatment efficacy of a dendritic cell-derived immunotherapeutic for prostate cancer comprises the steps of (a″) administering to a normal animal other than human a cancer cell line expressing the human prostate cancer-specific antigen to induce cancer in the normal animal; (b″) administering to the animal with cancer dendritic cells to be analyzed; and (c) determining the treatment efficacy of the dendritic cells as immunotherapeutics for prostate cancer by measuring the formation or growth of cancer cells originated from the cancer cell line in the animal.

The present method for analyzing the prevention efficacy of a dendritic cell-derived immunotherapeutic for prostate cancer comprises the steps of (a′) administering to a normal animal other than human dendritic cells to be analyzed; (b′) administering to the animal the cancer cell line expressing the human prostate cancer-specific antigen to induce cancer in the animal; and (c) determining the prevention of the dendritic cells as immunotherapeutics for prostate cancer by measuring the formation or growth of cancer cells originated from the cancer cell line in the animal.

The present invention provides firstly a successful protocol for analyzing the efficacy of a human dendritic cell-derived immunotherapeutic for prostate cancer using animal models. According to conventional technologies, animal models have not yet been provided for such analysis.

In the present invention, animals used include any animal species except for human, preferably mammals, more preferably rodents, still more preferably a mouse (Mus musculus), and most preferably Balb/c mouse. The term used herein “normal animal” refers to animals having not cancer.

According to the present method, an antigen used to establish a cancer cell line expressing a human prostate cancer-specific antigen includes any antigen expressed in human prostate cancer cells (Schmid, H. P. et al., Observations on the doubling time of prostate cancer: the use of serial prostate-specific antigen in patients with untreated disease as a measure of increasing cancer volume. Cancer 71:2031 (1993); Tjoa, B. A. et al., Evaluation of phase I/II clinical trials in prostate cancer with dendritic cells and PSMA peptides. Prostate 36:39 (1998)). Preferably, the human prostate cancer-specific antigen is PCA (prostate cancer antigen), PSCA (prostate stem cell antigen), PSA (prostate-specific antigen), PAP (prostate acid phosphatase) or PSMA (prostate-specific membrane antigen), more preferably PAP, PSA or PCA, still more preferably PAP or PSA, most preferably PAP. The human prostate cancer-specific antigens may comprise natural-occurring full length amino acid sequences as well as their partial sequences. Preferably, the antigen useful in this invention comprises an amino acid sequence spanning amino acids 30-204 of SEQ ID NO:1 for PCA, an amino acid sequence spanning amino acids 23-93 of SEQ ID NO:2 for PSCA, an amino acid sequence spanning amino acids 1-386 of SEQ ID NO:3 for PSA or an amino acid sequence spanning amino acids 1-261 of SEQ ID NO:4 for PSA or an amino acid sequence spanning amino acids 1-707 of SEQ ID NO:5 for PSMA.

The cancer cell lines used in cancer induction in normal animals may be derived from various animals. Preferably, the cancer cell line is allogeneic or syngeneic to the recipient animal, more preferably syngeneic to the recipient animal. According to a preferred embodiment, a mouse is used as normal animals and a mouse-derived cancer cell line is used as cancer cell lines. More preferably, Balb/c mouse is used as normal animals and a Balb/c-derived cancer cell line is used as cancer cell lines.

According to a preferred embodiment, the cancer cell line expressing the human prostate cancer-specific antigen is not originated from prostate cancer. In the meantime, a mouse derived prostate cancer cell line (C57BL/6 mouse derived RM cell line) has been suggested; however, it has not been elucidated to express prostate cancer-specific antigens described above. Therefore, dendritic cell-derived immunotherapeutics for prostate cancer could not be evaluated using the cancer cell line, which is unsuitable in the present invention.

The cancer cell lines useful in this invention include renal cancer cell lines (e.g., RENCA), gastric cancer cell lines, brain cancer cell lines, lung cancer cell lines, breast cancer cell lines, ovary cancer cell lines, liver cancer cell lines, bronchial cancer cell lines, nasopharyngeal cancer cell lines, laryngeal cancer cell lines, pancreatic cancer cell lines, bladder cancer cell lines, colon cancer cell lines and cervical cancer cell lines. Where renal cancer cell lines are used as cancer cell lines and Balb/c mice are used as recipient animals, a syngeneic renal cancer cell line, RENCA, is the most suitable in this invention.

Cancer cells other than prostate cancer cells are transformed with nucleotide sequences encoding human prostate-specific antigens and then used in the present invention. Human prostate cancer-specific antigen-encoding nucleotide sequences may comprise natural-occurring full length nucleotide sequences as well as their partial sequences. Preferably, the nucleotide sequence encoding human prostate cancer-specific antigens useful in this invention comprises a nucleotide sequence encoding an amino acid sequence spanning amino acids 30-204 of PCA, an amino acid sequence spanning amino acids 23-93 of PSCA, an amino acid sequence spanning amino acids 1-386 of PAP, an amino acid sequence spanning amino acids 1-261 of PSA, or an amino acid sequence spanning amino acids 1-707 of PSMA. More preferably, the nucleotide sequence encoding human prostate cancer-specific antigens comprises a nucleotide sequence of nucleotides 88-612 of SEQ ID NO:6 for PCA, a nucleotide sequence of nucleotides 67-279 of SEQ ID NO:7 for PSCA, a nucleotide sequence of nucleotides 1-1158 of SEQ ID NO:8 for PAP, a nucleotide sequence of nucleotides 1-783 of SEQ ID NO:9 for PSA, or a nucleotide sequence of nucleotides 1-2121 of SEQ ID NO:10 for PSMA.

The nucleotide sequences coding for human prostate cancer-specific antigens may be prepared by a variety of methods. For instance, total RNA is isolated from human-derived prostate cancer cell line (e.g., LNCaP.FGC), from which cDNA molecules are synthesized using primers designed by referring to known nucleotide sequences encoding human prostate cancer-specific antigens. The cDNA molecules synthesized thus are cloned into suitable expression vectors for animal cells (e.g., pcDNA3.1(+)) and introduced into cancer cells (e.g., renal cancer cell, RENCA) other than prostate cancer cells. Among cells, transformed cancer cells expressing human prostate cancer-specific antigens are selected and used to establish cancer cell line expressing human prostate cancer-specific antigens.

As described above, human prostate cancer-specific antigens-expressing cancer cell lines are established for the first time by the present inventors. The cancer cell line expressing a human prostate cancer-specific antigen processes the human prostate cancer-specific antigen expressed and displays the processed antigen molecule on its surface through Major Histocompatibility Complex I. As results, the cancer cell line permits to be recognized by T cells specific to the human prostate cancer antigen.

Dendritic cells to be analyzed in this invention may be prepared by various protocols known to one of skill in the art. For example, dendritic cells are obtained from monocytes, hematopoietic progenitor cells or bone marrow cells.

The preparation process for dendritic cells using bone marrow cells are exemplified as follows: Bone marrow cells are isolated from a femur and tibia of mice and the cultured in media containing suitable cytokines (e.g., IL-4 and GM-CSF) for the differentiation to dendritic cells. The immature dendritic cells obtained thus are pulsed with a human prostate cancer-specific antigen and then cultured in media containing suitable cytokines for maturating dendritic cells, which serve as samples to be analyzed. The pulsing becomes very effective when CTP (cytoplasmic transduction peptide)-conjugated antigens are used. The CTP molecule delivers antigens into cytoplasm not nucleus, which permits dendritic cells to present more effectively antigens on their surface through Major Histocompatibility Complex Class I (MHC I) molecules. The descriptions of CTP molecules are also found in WO 03/097671, the teachings of which are incorporated herein by reference.

The dendritic cells to be analyzed may be administered into animals via various routes, preferably intravenous injection or subcutaneous injection, most preferably subcutaneous injection. The cancer cell lines expressing human prostate cancer-specific antigens may be administered into normal animals via various routes, preferably intravenous injection or subcutaneous injection, most preferably subcutaneous injection (Fong, L. et al., Dendritic cells injected via different routes induce immunity in cancer patients. J. Immunol. 166:4254. (2001)).

The dendritic cells in the step (a) are administered into animals, e.g. mice in a dose of 1×10⁴−1×10⁵ cells, preferably 1×10⁵−1×10⁷ cells and more preferably about 1×10⁶ cells. It is preferred that the administration of dendritic cells is performed twice in a suitable time interval (e.g., one week).

The cancer cell line in the step (a) are administered into animals, e.g. mice in a dose of 1×10⁴−1×10⁸ cells, preferably 1×10⁵−1×10⁷ cells and more preferably about 1×10⁶ cells.

Based on knowledge available to one of skill in the art, it could be generally believed that when cancer cell lines expressing human prostate cancer-specific antigens are administered to animal except for human, they are very likely to be eliminated by immune reactions in animals. Surprisingly, according to the present invention, human prostate cancer antigen-expressing cancer cell lines administered to animal except for human induce the formation of cancerous tissues in animals, which enables the present method to be successfully performed.

The administration route and dose of cancer cell lines in the step (a) described above can be also applied to the step (b).

According to the present invention, (i) the human prostate cancer-specific antigen used to pulse dendritic cells in the step (a′) and (ii) the human prostate cancer-specific antigen expressed in the cancer cell line of the step (b′) are originated from the same one antigen. For instance, where the human prostate cancer-specific antigen used to pulse dendritic cells in the step (a′) is PAP, the human prostate cancer-specific antigen expressed in the cancer cell line of the step (b′) expresses is also PAP. Therefore, cytotoxic T lymphocytes induced by dendritic cells presenting PAP recognize cancer cell lines expressing PAP, resulting in the lysis of cancer cell lines.

In the final step of the present invention, the formation or growth of cancer cells in animals are measured to determine the prevention or treatment efficacy of the dendritic cells as immunotherapeutics for prostate cancer. The formation or growth of cancer cells in animals can be evaluated with naked eye or using devices such as calipers. Where the further formation or growth of cancer cells are observed, it can be determined that dendritic cells of interest as immunotherapeutics possess the prevention or treatment efficacy for prostate cancer.

For executing the prevention or treatment of prostate cancer using dendritic cells in a clinical scale, it is prerequisite to verity the efficacy and safety of dendritic cells in animal models. The present invention allows for animal model-based evaluation of dendritic cells as immunotherapeutics. Dendritic cells selected by the present invention become promising candidates as immunotherapeutics for prostate cancer.

In another aspect of this invention, there is provided a mouse-derived renal cancer cell line (RENCA) expressing a human prostate cancer-specific antigen, characterized in that the human prostate cancer-specific antigen is PCA (prostate cancer antigen), PSCA (prostate stem cell antigen), PSA (prostate-specific antigen), PAP (prostate acid phosphatase) or PSMA (prostate-specific membrane antigen) and the renal cancer cell line is not a prostate cancer cell-derived one.

The renal cancer cell line (RENCA) expressing a human prostate cancer-specific antigen of this invention has been firstly developed by the present inventors for establishing prostate cancer animal models.

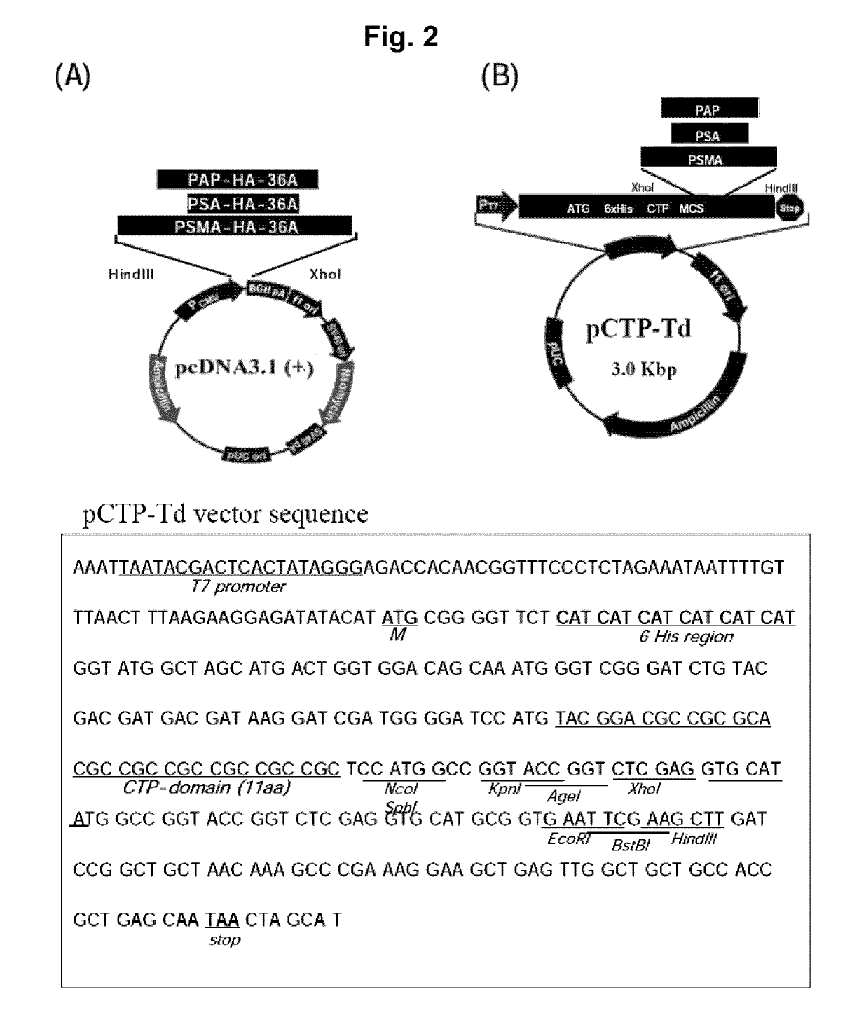

The renal cancer cell line of this invention is prepared by transforming with a nucleotide sequence encoding PCA, PSA or PAP. Human prostate cancer-specific antigen-encoding nucleotide sequences may comprise natural-occurring full length nucleotide sequences as well as their partial sequences. Preferably, the renal cancer line is transformed with a vector containing a nucleotide sequence encoding an amino acid sequence spanning amino acids 30-204 of PCA, an amino acid sequence spanning amino acids 1-386 of PAP or an amino acid sequence spanning amino acids 1-261 of PSA. More preferably, the PCA-encoding sequence is set forth in nucleotides 88-612 of SEQ ID NO:6 for PCA, the PAP-encoding sequence is set forth in nucleotides 1-1158 of SEQ ID NO:8, and the PSA-encoding sequence is set forth in nucleotides 1-783 of SEQ ID NO:9. Most preferably, the cancer cell line expressing human prostate cancer-specific antigens is transformed with pcDNA3.1(+)-Tag/PAP, pcDNA3.1(+)-Tag/PSA or pcDNA3.1(+)-Tag/PCA as depicted in FIG. 2. In FIG. 2, “PCA”, “PAP” and “PSA” represent nucleotides 88-612 of SEQ ID NO:6, nucleotides 1-1158 of SEQ ID NO:8 and nucleotides 1-783 of SEQ ID NO:9, respectively.

The cancer cell line expressing human prostate cancer-specific antigens processes the human prostate cancer-specific antigen expressed and presents the processed antigen molecule on its surface through Major Histocompatibility Complex I. As results, the cancer cell line permits to be recognized by T lymphocytes specific to the human prostate cancer antigen.

In still another aspect of this invention, there is provided a mouse prostate cancer model, characterized in that the mouse model has a cancer formed by inoculating the renal cancer cell line of this invention expressing the human prostate cancer-specific antigen, and the metastasis or growth of the cancer formed in the mouse model is inhibited by the treatment of dendritic cells pulsed with the human prostate cancer-specific antigen.

The mouse prostate cancer model bears a cancer formed by inoculating the mouse renal cancer cell line expressing human prostate cancer-specific antigen and allows for the evaluation of dendritic cells as immunotherapeutics for prostate cancer. Mouse models have not been yet suggested to evaluate the prevention and treatment efficacy for prostate cancer.

According to a preferred embodiment, the renal cancer cell line injected into the mouse is syngeneic to the mouse. According to another preferred embodiment, the mouse prostate cancer model is used for performing the present method to analyze the prevention and treatment efficacy of dendritic cells for prostate cancer described hereinabove. And according to still another preferred embodiment, the mouse model of this invention is Balb/c mouse syngeneic to the injected cancer cell line.

The following specific examples are intended to be illustrative of the invention and should not be construed as limiting the scope of the invention as defined by appended claims.

### EXAMPLES

### Example 1

### Preparation of Mouse Cell Lines Expressing Human-Derived Prostate Cancer Antigens

### Example 1-1

### Construction of Expression Vectors for Human-Derived Prostate Cancer Antigens

### (a) Culture of Human-Derived Prostate Cancer Cell Line LNCaP.FGC

The LNCaP.FGC used in this experiment is human-derived prostate cancer cell line expressing human prostate cancer-specific antigens such as PCA (prostate cancer antigen), PSCA (prostate stem cell antigen), PSA (prostate-specific antigen), PAP (prostate acid phosphatase) or PSMA (prostate-specific membrane antigen), and was obtained from the Korean Cell Line Research Foundation. The prostate cancer cell line was cultured and maintained in RPMI-1640 medium (Gibco BRL) containing 10% FBS. Cultured cells were treated with trypsin-EDTA for 1 min to obtain non-adherent single cells and then subcultured to 80% confluency. The subculturing was carried out 2-3 times a week.

(b) Preparation of cDNA PCR Products of PCA, PSCA, PSA, PAP and PSMA in LNCaP

Prior to harvesting LNCaP, cells were subcultured 2-3 times to 60% confluency and trypsinized, followed by harvesting cells. Total RNA was extracted using Trizol (Gibco BRL) from cells harvested and subjected to isopropanol precipitation and 70% ethanol washing for purification. For synthesizing cDNA, a mixture of 10 μg of total RNA and 1 μg of oligo (dT)₁₂₋₁₈ primer were denatured for 5 min at 65° C. and transferred on ice, to which reverse transcriptase buffer, 10 mM DTT, 1 mM dNTP mixture and 20 units RNAs in were added. The reactant mixture was prereacted for 2 min at 42° C. and then underwent reverse transcription using 200 units MMLV (Molony Murine Leukemia Virus) reverse transcriptase (Invitrogen, Inc.) for 60 min at 42° C. After the completion of reactions, the reactions were kept to stand for 15 min at 70° C. to inactivate the enzyme. PCR reactions were carried out using cDNA molecules synthesized as templates for amplifying cDNA molecules of PCA, PSCA, PAP, PSA and PSMA. The primer sequences used are summarized in Tables 1a and 1b.

TABLE 1a

Primers for Cloning into Prokaryotic Expression
Vectors

| Target | | |
|---|---|---|
| gene | Primer | Sequence |
| PAP | PAP-XhoI/s | 5′-TACCCCTCGAGAAGGAGTTGAAGTTTGT |
| | | GACT-3′ |
| PAP | PAP-Hind/as | 5′-TACCCAAGCTTTTAATCTGTACTGTCCT |
| | | CAGTAC-3′ |
| PSA | PSA-XhoI/s | 5′-TACCCCTCGAGCCCCTCATCCTGTCTCG |
| | | G-3′ |
| PSA | PSA-Hind/as | 5′-TACCCAAGCTTTTAGGGGTTGGCCACGA |
| | | TGGT-3′ |
| PSMA | PSMA-XhoI/as | 5′-TACCCCTCGAGGTTGTTCATGAAATTGT |
| | | GAGG-3′ |
| PSMA | PSMA-Hind/as | 5′-TACCCAAGCTTTTATCCTGGGAATGACT |
| | | CCCCT-3′ |
| PCA | PCA-XhoI/s | 5′-TTGACCTCGAGCACGCGCCCTGGGACAA |
| | | C-3′ |
| PCA | PCA-Hind/as | 5′-TCTAAAAGCTTCTAGAGGCTGCAGGCCT |
| | | CCTG-3′ |
| PSCA | PSCA-XhoI/s | 5′-TTGACCTCGAGCAGCCAGGCACTGCCCT |
| | | G-3′ |
| PSCA | PSCA-Hind/as | 5′-TCTAAAAGCTTCTACGGCTGCAGGGCAT |
| | | GG-3′ |

TABLE 1b

Primers for Cloning into Eukaryotic Expression
Vectors

| Target | | |
|---|---|---|
| gene | Primer | Sequence |
| PAP | PAP-Hind/s | 5′-TACCCAAGCTTCGCCACCATGGGTAGAG |
| | | CTGCACCCCTCCTC-3′ |
| PAP | PAP-XhoI/as | 5′-TACCCCTCGAGCCAGCATAATCTGGAAC |
| | | ATCATATGGATAATCTGTACTGTCCTCAGTA |
| | | CC-3′ |
| PSA | PSA-Hind/s | 5′-TACCCATGCTTCGCCACCATGGGTGTCC |
| | | CGGTTGTCTTCCTCA-3′ |
| PSA | PSA-XhoI/as | 5′-TACCCCTCGAGCCAGCATAATCTGGAAC |
| | | ATCATATGGATAGGGGTTGGCCACGATGG |
| | | T-3′ |
| PSMA | PSMA-Hind/s | 5′-TACCCAAGCTTCGCCACCATGGATTGGA |
| | | ATCTCCTTCACGAAAC-3′ |
| PSMA | PSMA-XhoI/as | 5′-TACCCCTCGAGCCAGCATAATCTGGAAC |
| | | ATCATATGGATATCCTGGGAATGACTCCCC |
| | | T-3′ |
| PCA | PCA-Hind/s | 5′-TTGACAAGCTTCGCCACCATGGGAGCCC |
| | | CGCTCGCCGTAG-3′ |
| PCA | PCA-XhoI/as | 5′-TCTAACTCGAGCCAGCATAGTCTGGGAC |
| | | GTCATATGGATAGAGGCCTGCAGGCCT CCT |
| | | G-3′ |
| PSCA | PSCA-Hind/s | 5′-TTGACAAGCTTCGCCACCATGGGAGCTG |
| | | TGCTGCTTGCCCTGT-3′ |
| PSCA | PSCA-XhoI/as | 5′-TCTAACTCGAGCCAGCATAGTCTGGGAC |
| | | GTCATATGGATAGTTGCACAAGTCGGTGTCA |
| | | C-3′ |

In Table 1b, the underlines denote HA (hemagglutinin)-Tag sequence.

Using primer sets (Bionics, Inc.) in Table 1a and PCR polymerase (Intron Biotech, Inc.), PCR amplifications were conducted for obtaining DNA fragments, PAP (1062 bp), PSA (729 bp), PSMA (942 bp), PCA (525 bp) and PSCA (213 bp), for expressing in prokaryotic cells under the following thermal conditions: 25 cycles of 30 sec at 94° C., 30 sec at 52° C., and 50 sec at 72° C. Likely, for obtaining DNA fragments, PAP (1185 bp), PSA (810 bp), PSMA (2148 bp), PCA (552 bp) and PSCA (240 bp), for expressing in eukaryotic cells, PCR amplifications were conducted using primer sets in Table 1b. For making it feasible to detect proteins expressed in eukaryotic cells, 36A Tag sequence developed by CreaGen, Inc. (Korea) was introduced to the amplified nucleotide sequences. Primers for introducing Tag sequence are Tag-XhoI/s(5′-ACCCTCGAGGTCCATGACCGGAGGTCAGC AGATGGGTCGCGACCTGTACGACGA-3′) and Tag-XbaI/as(5′-ACCTCTAGATT AGCTTCCCCATCTGTCCTTGTCGTCATCGTCGTACAGGTCGCG-3′). Tag DNA fragments were prepared by PCR amplification under the thermal conditions: 1 cycle of 30 sec at 94° C., 30 sec at 52° C., and 5 min at 72° C.

The amino acid sequence of 36A Tag is SMTGGQQMGRDLYDDDDKDRWGS and its nucleotide sequence is TCC ATG ACC GGA GGT CAG CAG ATG GGT CGC GAC CTG TAC GAC GAT GAC GAC AAG GAC AGA TGG GGA AGC.

(c) Cloning Prostate Cancer Antigen cDNA into Expression Vectors (pcDNA3.1(+) Vector and pCTP Vector)

To introduce into vector (pcDNA3.1(+); Invitrogen) 36A Tag system for evaluating expression, Tag DNA fragments prepared were digested using XhoI/XbaI and ligated to pcDNA3.1(+), producing pcDNA3.1(+)-Tag vector. Each of DNA fragments for human prostate cancer antigens was digested using HindIII/XhoI and cloned into pcDNA3.1(+)-Tag vector, followed by confirming cloned sequences by sequencing (see FIG. 2). To obtain recombinant prostate cancer antigens in prokaryotic cells, pCTP-Td vector was used.

pCTP-Td vector was constructed by genetically manipulating pTAT-HA vector (kindly provided by Dr. S. Dowdy at the Washington University, H. Nagahara et al., Nature Med. 4:1449 (1998)). pTAT-HA vector was digested for 2 hr at 37° C. using BamHI and NcoI to eliminate Tat domain and HA-tag domain, and then purified using Gel extraction kit (Nucleogen). For preparing CTP DNA fragments (nucleotide sequence, TAC GGA CGC CGC GCA CGC CGC CGC CGC CGC CGC; amino acid sequence, YGRRARRRRRR), a forward primer (5′-GAT CCA TGT ACG GAC GCC GCG CAC GCC GCC GCC GCC GCC GCT C-3′) and a reverse primer (5′-CAT GGA GCG GCG GCG GCG GCG GCG TGC GCG GCG TCC GTA CAT G-3′) were synthesized (Bionics, Inc.), heated for 5 min at 95° C. and cooled to room temperature at a rate of 1° C./min to prepare double stand. Primers were designed to permit CTP DNA fragments to have cohesive end for BamHI and NcoI. After purification of annealed CTP DNA fragments using PCR purification kit (Necleogen), they were ligated to vectors using T4 DNA ligase (Roche) and then transformed into E. coli JM109 (Stratagene), yielding recombinant plasmid, pCTP-Td vector. The construction of pCTP-Td vector was confirmed by both restriction analysis using BamHI and NcoI and sequencing (Solgent, Korea).

Each of DNA fragments for human prostate cancer antigens was digested using HindIII/XhoI and cloned into pCTP vector (see FIG. 2).

DNA sequencing was undertaken for the nucleotides sequences cloned. It was verified that the amino acid sequences encoded by the cloned sequences had 100% identity to known amino acid sequences of PCA, PSCA, PAP, PSA and PSMA (Blast 2 sequence search). The sequences introduced into prokaryotic expression vectors are lack of sequences corresponding to N-terminal signal peptide and transmembrane domain adjacent to C-terminal. The nucleotide sequences into prokaryotic expression vectors encode amino acids 30-204 of PCA (175 aa), amino acids 23-93 of PSCA (71 aa), amino acids 33-386 of PAP (354 aa), amino acids 19-261 of PSA (243 aa) and amino acids 394-707 of PSMA (314 aa). In the meantime, the nucleotide sequences into eukaryotic expression vectors encode amino acids 30-204 of PCA (175 aa), amino acids 23-93 of PSCA (71 aa), amino acids 1-386 of PAP (386 aa), amino acids 1-261 of PSA (261 aa) and amino acids 1-707 of PSMA (707 aa).

### Example 1-2

### Establishment of Human-Derived Prostate Cancer Antigen-Expressing Mouse Cell Lines

### (a) Analysis of Antigen Expression in Prostate Cancer Antigen-Expressing Mouse Cell Lines

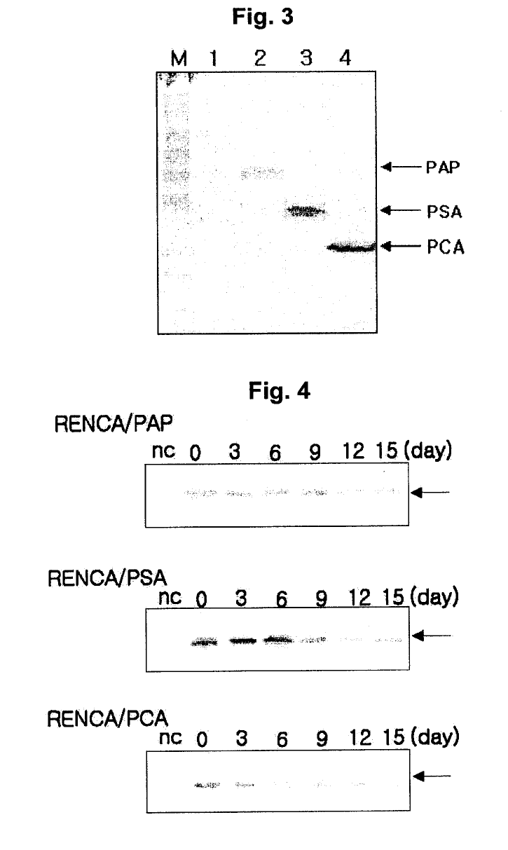

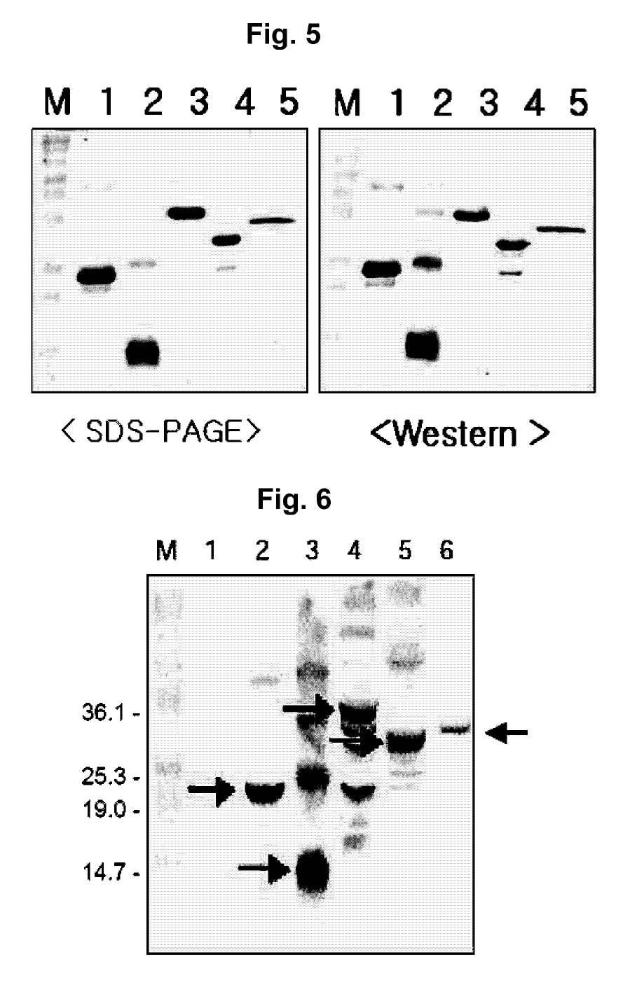

To prepare prostate cancer antigen-expressing cell lines, pcDNA3.1(+)-Tag/prostate cancer antigen (PAP, PSA or PCA) vectors were transformed into mouse renal cancer cell line RENCA cells (the Korean Cell Line Research Foundation). On the day before transformation, 5×10⁵ cells/well were plated into 6-well plate and then transformed with 2 μg of the recombinant vector using GenePorter (Gene therapy system). 48 hours later, cells were treated with 800 μg/ml of G418 (DUCHEFA, Haarlem, Netherland) to select viable cells. Cells selected were proliferated and harvested and in turn the expression pattern of antigens was verified by Western blotting analysis. Harvested cells were washed twice with PBS, heated in protein sample buffer and centrifuged to remove genomic DNA molecules, followed by SDS-PAGE for supernatant isolation. Protein bands resolved were transferred to a nitrocellulose membrane using semi-dry transfer blotter (Bio-Rad) and incubated with a primary antibody, Tag antigen-specific monoclonal antibody (CreaGen, Inc.) and a secondary antibody AP (alkaline phosphatase)-conjugated anti-mouse IgG (Sigma). The bands were visualized using AP reaction solution (Promega) containing NBT/BCIP. As shown in FIG. 3, proteins of interest were successfully expressed in three cell lines harboring expression vectors for antigens.

Of prostate cancer antigen-expressing cell lines established, PAP-expressing cell line was denoted as “RENCA/PAP”, deposited on May 23, 2005 in International Depository Authority, the Korean Collection for Type Cultures and was given accession number KCTC 10808BP.

### (b) Evaluation of Cell Line Stability in View of Antigen Expression

To verify whether recombinant cell lines established maintain the antigen expression potential in the absence of antibiotics (G418) when injected into mice, cell lines were subcultured in a medium with no G418 and examined, so that the stable expression of introduced foreign sequences were checked. Each of cell lines (RENCA/PAP, RENCA/PSA and RENCA/PCA) was cultured in the absence of G418, 1×10⁶ cells were harvested every three days and then their antigen expression potential was examined by Western blotting analysis using anti-Tag monoclonal antibody. As shown in FIG. 4, the antigen expression potential is maintained for 15 days in all of three cell lines maintain although it decreases slightly with the lapse of time. Consequently, it could be appreciated that human prostate cancer antigen-expressing cell lines prepared in Examples are very useful in the construction of mouse prostate cancer models.

### Example 2

### Purification of Recombinant CTP-Conjugated Proteins for Pulsing Dendritic Cells and Measurement of Transduction Potential

### Example 2-1

### Expression and Purification of Recombinant CTP-Conjugated Prostate Cancer Antigens

E. coli BL21Gold(DE3) competent cells (Stratagene) were transformed with recombinant pCTP-Td vectors carrying cDNA for each prostate cancer antigen to prepare transformants according to Hanahan method and cultured in ampicillin-LB medium. The transformants cultured were centrifuged, washed with PBS and harvested, followed by analyzing prostate cancer antigen expression on 12% SDS-PAGE. Following the expression, recombinant proteins CTP-PCA, CTP-PSCA, CTP-PSA, CTP-PAP and CTP-PSMA were purified through a column of Ni⁺-NTA resin (Qiagen). The proteins analyzed were shown to have a molecular weight higher by about 6 kDa, which is ascribed to non-genome sequence originated from vectors. In other words, CTP-PCA shows a molecular weight of about 24 kDa, CTP-PSCA of about 14 kDa, CTP-PAP of about 45 kDa, CTP-PSA of about 33 kDa and CTP-PSMA of about 41 kDa.

The CTP-PSMA expressed corresponds to the C-terminal of PSMA. Since the N-terminal signal sequence of PSMA was not revealed in a motif prediction analysis, the expression of the region corresponding amino acids 2-707 (706 aa) was primarily attempted. Unlikely to eukaryotic cells, the expression of PSMA was not observed in E. coli. Thereafter, either the N-terminal region (2-393) or C-terminal region (394-707) was cloned into pCTP vector and expressed. As results it was revealed that only the C-terminal region was shown to be expressed. These results urge us to reason that hydrophobic residues concentrated at the N-terminal region would inhibit its expression. Accordingly, we used the C-terminal region of PSMA for preparing vaccines as immunotherapeutics.

### Example 2-2

### Transduction Potential of Recombinant CTP-Prostate Cancer Antigens

To elucidate the transduction potential of recombinant proteins, cells were incubated with each of five recombinant proteins (CTP-PCA, CTP-PSCA, CTP-PAP, CTP-PSA and CTP-PSMA). 1×10⁵ mouse splenocytes were cultured in a 6-well plate and incubated with 50 μg/well of the recombinant protein. 24-hr later, cells were collected and trypsinized to remove proteins attached on their surface. Then, cells were lyzed using a sonicator and resolved by PAGE, followed by Western blotting using anti-His6-tag monoclonal antibody (Qiagene).

As represented in FIG. 6, CTP-PCA, CTP-PSCA, CTP-PAP, CTP-PSA and CTP-PSMA show the bands of about 24 kDa, about 14 kDa, about 33 kDa, about 40 kDa and about 41 kDa, respectively. PSCA shows multi-band pattern since it forms dimmer or multimer. Therefore, it could be understood that recombinant CTP-conjugated prostate cancer antigens are successfully introduced into cells.

### Example 3

### Establishment of Animal Prostate Caner Models

### Example 3-1

### Evaluation of Formation and Growth of Cancer Caused by Prostate Cancer Antigen-Expressing Cell Lines in Mice

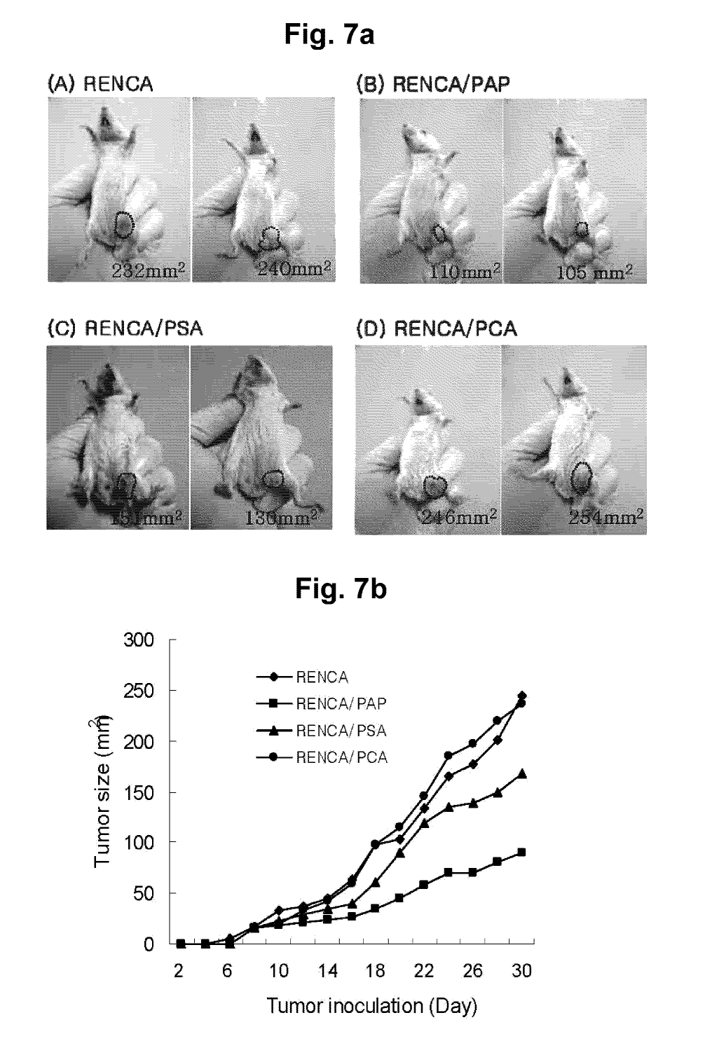

We examined the formation and growth of cancer caused by mouse cell lines expressing human prostate cancer antigens. Cell lines prepared hereinabove were injected into femurs of 6-week-old Balb/c mouse (Daehan Biolink, Inc., Korea). The recombinant cell lines expressing human prostate cancer antigens were cultured and maintained in RPMI medium containing 10% FBS and 500 μg/ml G418. Cells at optimal growth state were washed 2-3 times with PBS, treated with trypsin-EDTA for single cell isolation, and suspended at 1×10⁶ cells/100 μl in PBS. 100 μl of the suspension were subcutaneously inoculated into mice. Following the inoculation of cell lines, the formation of solid cancer was observed in a time interval of 2 days. The size of solid cancer was measured using calipers. As represented in FIG. 7a, all mice inoculated with prostate cancer cell lines were found to bear solid cancer. Interestingly, a lower dose of cells, e.g. 1×10⁵ cells could cause tumorigenesis and cancer formed in mice were not extinguished by immune response to heterologous antigens.

FIG. 7b shows the growth rate of cancer. The recombinant RENCA/PAP cell line shows a growth rate of cancer lower than other cell lines and the RENCA/PCA cell shows a growth rate of cancer similar to RENCA. It would be understood that the expression of PAP is made in the highest level and in turn elicits the strongest immune responses.

These results demonstrate that the tumorigenesis in mice caused by cells expressing human prostate cancer antigens could not be prevented by immune responses to heterologous antigens. Accordingly, it could be determined that prostate cancer mouse models are successfully established by the present invention and allows for evaluating the prevention and treatment efficacy of dendritic cell-based vaccines.

### Example 4

### Analysis of Anti-Cancer Efficacy of Dendritic Cells

### Example 4-1

### Prevention Efficacy of Dendritic Cell-Based Vaccine (Prevention Model)

To investigate whether dendritic cell-based vaccines can prevent prostate cancer, mice were immunized twice with dendritic cells pulsed with recombinant CTP-conjugated prostate cancer antigens and challenged with cancer cell lines expressing prostate cancer-specific antigen, followed by examining the formation of solid cancer and pulmonary metastasis.

Mice dendritic cells were prepared by differentiating bone marrow cells of a femur and tibia into dendritic cells. The both ends of a femur and tibia were dissected, from which bone marrow cells were extracted and collected into a 50 ml tube. Bone marrow cells collected were suspended in 0.83% ammonium chloride solution to remove red blood cells, and cultured in a dendritic cell production medium (RPMI-1640 medium containing 10% FBS, 10 ng/ml mouse recombinant IL-4 and 10 ng/ml mouse GM-CSF) for 2 days. Non-adherent cells were removed to obtain only adherent cells on the bottom of tubes. Medium was changed with a fresh medium in a time interval of 2-3 days to prevent the deficiency of cytokines. On a 6 day of culture, immature dendritic cells were harvested and incubated with CTP-PSA, CTP-PCA or a 1:1 mixture of CTP-PSA and CTP-PCA. Immature dendritic cells were pulsed with 50 μg/ml of each antigen protein for 20 hr, to which 100 μg/ml of IFN-γ and 100 μg/ml of TNF-α as cytokines for DC maturation were added. 1×10⁶ cells of dendritic cells pulsed with antigens were subcutaneously injected into mice to elicit immune reactions to cancer. Immunization with dendritic cells was conducted twice in a time interval of 2 weeks. After one week of the second immunization, mice immunized by denritic cells pulsed with CTP-PAP, mice immunized by denritic cells pulsed with CTP-PSA, and mice immunized by denritic cells pulsed with CTP-PCA were subcutaneously challenged with 1×10⁶ cells/mouse of RENCA/PAP, RENCA/PSA and RENCA/PCA, respectively. The size of cancer (length×breadth) was measured every two days.

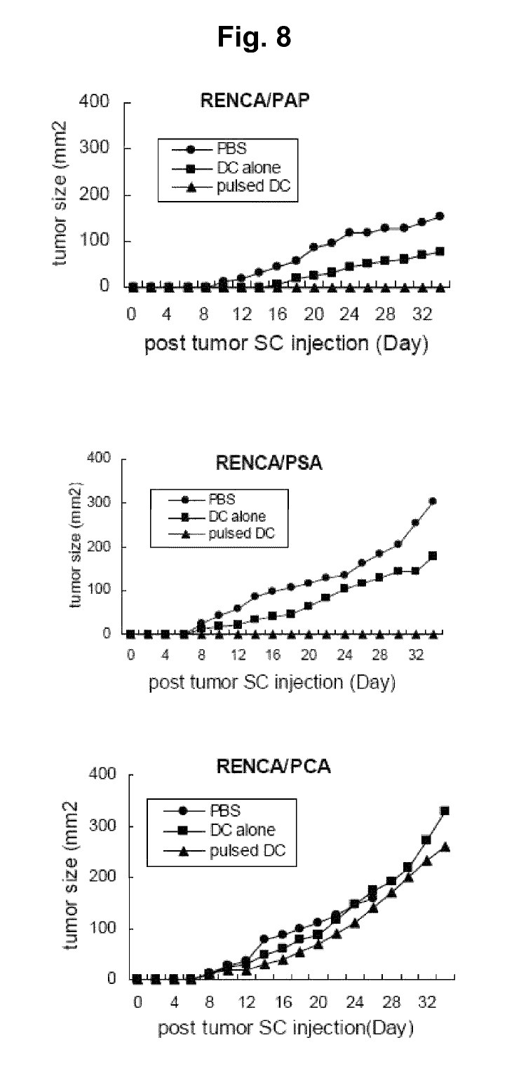

As shown in FIG. 8, mice immunized using dendritic cells pulsed with CTP-PAP or CTP-PSA antigen were shown to bear no tumor mass. In contrast, mice immunized using dendritic cells pulsed with CTP-PCA had cancerous tissues.

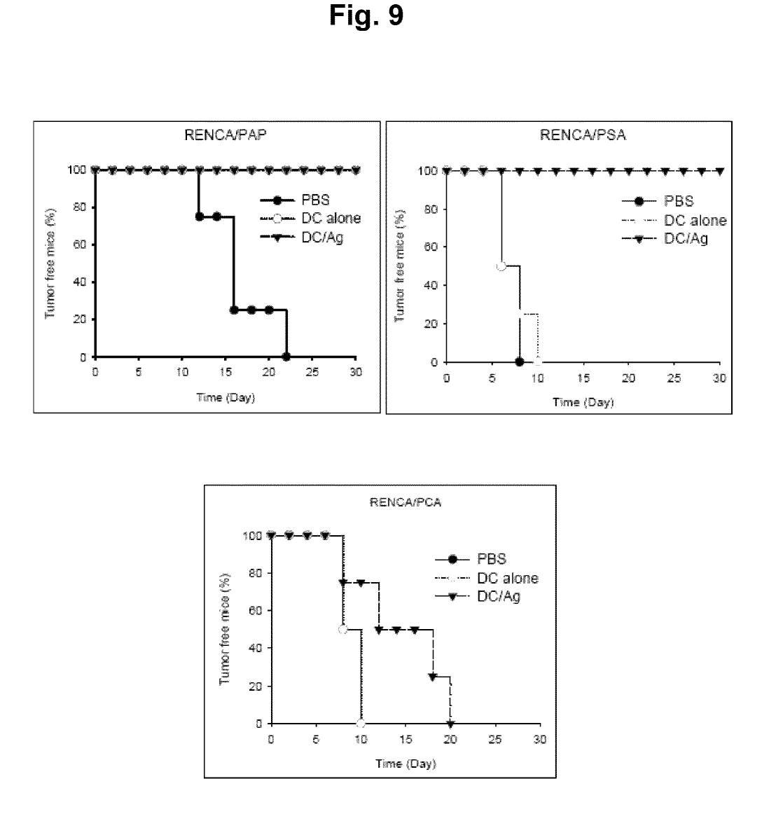

FIG. 9 graphically shows the cancer incidence in a cancer prevention model using dendritic cells. Among mice immunized with antigen-pulsed dendritic cells, mice challenged with RENCA/PAP or RENCA/PSA cell lines were found to exhibit 100% prevention efficacy for cancer. Mice challenged with RENCA/PCA cell line were shown to exhibit the prevention efficacy much lower than mice challenged with RENCA/PAP or RENCA/PSA, although tumorigenesis was retarded. These results are ascribed to a low antigenecity of PCA. Therefore, it is preferable that DC vaccines for prostate cancer are not prepared using PCA as a sole antigen.

### Example 4-2

### Inhibition of Prostate Cancer Metastasis by Dendritic Cell-Based Vaccine (Prevention Model)

Mice were immunized twice with dendritic cell-based vaccines as described hereinabove and injected via tail vein with 1×10⁶ cells/mouse of cell lines expressing prostate cancer antigens. 30 days later, mice were euthanized and pulmonary metastasis was evaluated.

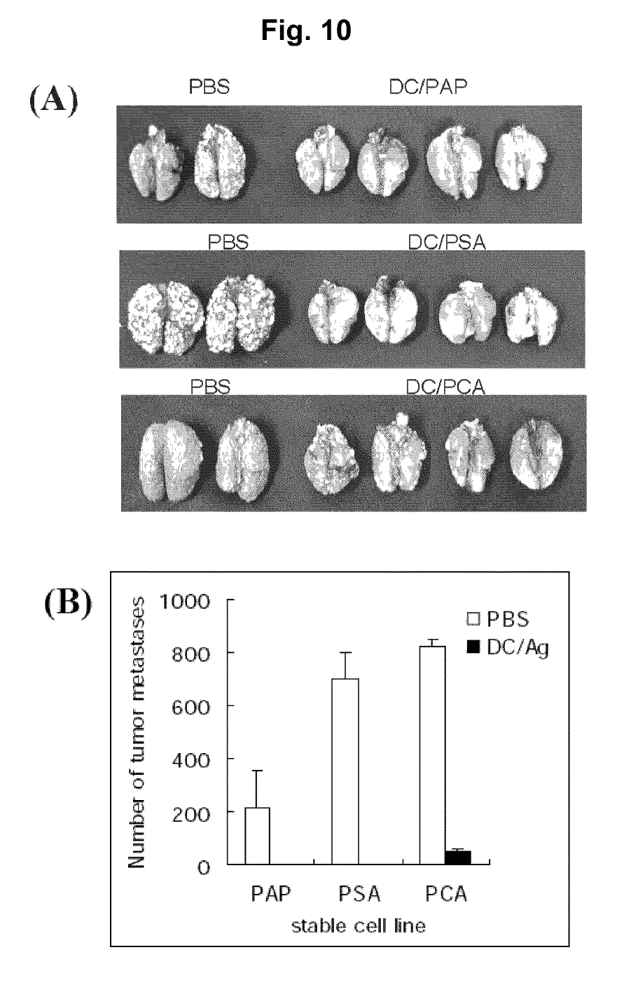

As shown in FIG. 10, mice immunized using dendritic cells pulsed with human prostate cancer antigens were shown to exhibit no pulmonary metastasis. In contrast, mice treated with either dendritic cells not pulsed or PBS were shown to elicit strong pulmonary metastasis. It could be understood that dendritic cell-based vaccines elicit strong immune reactions specific to cancer antigens and in turn inhibit the formation and metastasis of cancer.

### Example 4-3

### Treatment Efficacy of Dendritic Cell-Based Vaccine (Regression Model)

Mice were subcutaneously injected with 1×10⁶ cells/mouse of recombinant cell lines expressing prostate cancer antigen (PAP, PSA and PCA). After 3 days, mice were injected twice in a time interval of one week with 1×10⁶ cells/mouse of dendritic cells pulsed with CTP-conjugated antigens (CTP-PAP, CTP-PSA and CTP-PCA). Following the second administration of DC, the formation and growth of cancer were observed for one month in a time interval of 2 days.

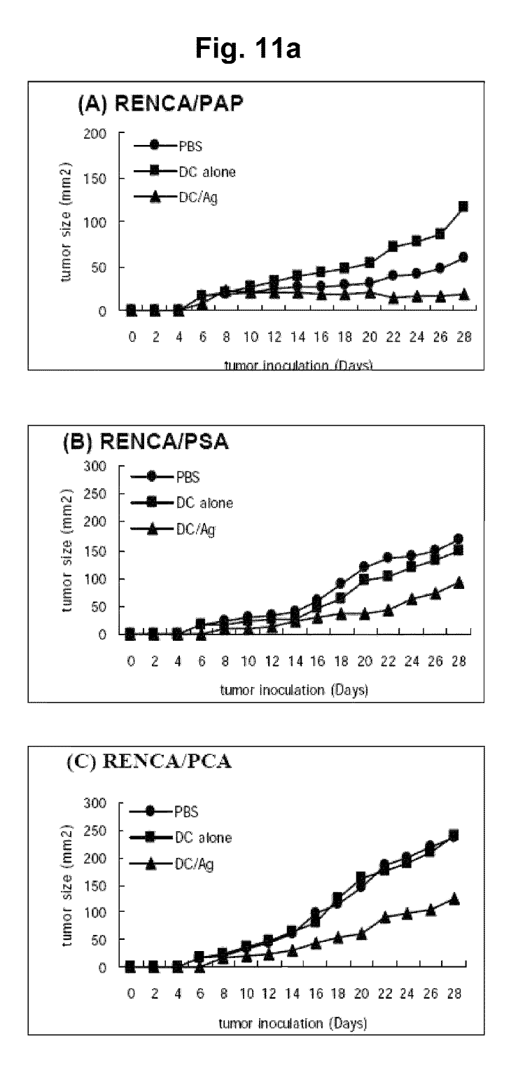

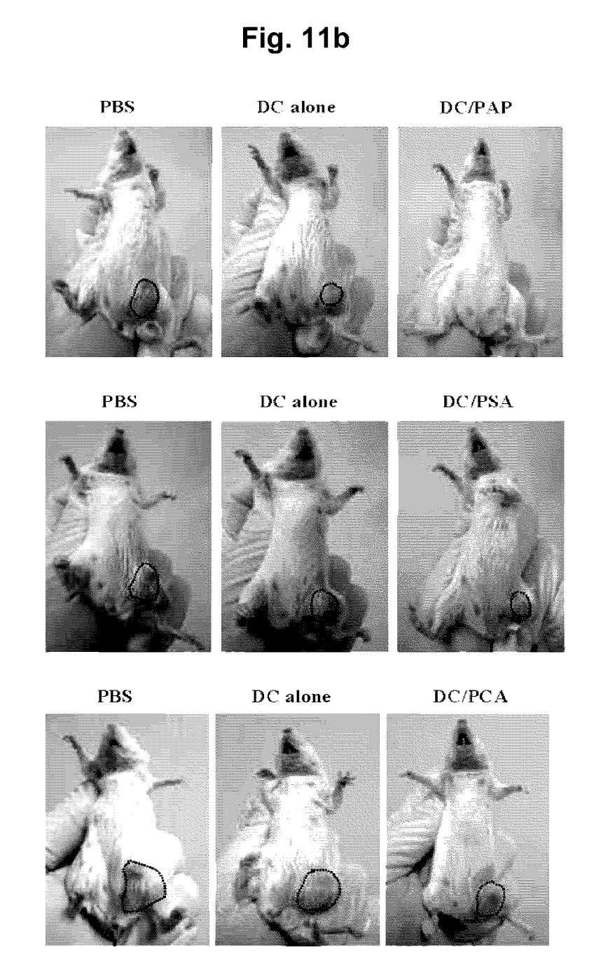

As represented in FIGS. 11a and 11b, the growth of cancer was inhibited in all of prostate cancer mouse models established using RENCA/PAP, RENCA/PSA and RENCA/PCA cell lines. In particular, the RENCA/PAP mouse model was shown to exhibit the strongest inhibition to tumorigenesis.

### Example 4-4

### Analysis of CTL Response Induced by Dendritic Cell-Based Vaccine

T cell proliferation and CTL activity were analyzed using splenocytes isolated from mice of Example 4-3. Each mouse was euthanized according to cervical dislocation method and spleen was isolated and stored in RPMI. Each spleen was passed through a 70 μm sieve and suspended tissues were then removed. The resultant was centrifuged to collect cells, after which were suspended in 0.83% ammonium chloride solution to remove red blood cells. The splenocytes prepared were passed through a nylon wool column to isolate T lymphocytes as effector cells, and mixed with APC (antigen presenting cells) at a ration of 5:1, followed by culturing for 5 days. APC was prepared 2 days prior to experiment. Separately, splenocytes were isolated from normal mice and treated for 3 hr with 3 μg/ml of Con-A. The stimulated cells were incubated with 50 μg/ml of each antigen protein (CTP-PAP, CTP-PSA and CTP-PCA) for 24 hr. The cell concentration was maintained at 1×10⁶ cells during culture. After 24-hr culture, cells were treated with mitomycin C for 40 min and washed three times to prepare APC. After 5-days of culture of effector T cells, effector T cells were isolated by removing dead cells with Histopaque (Sigma). The effector T cell and target cell were mixed at ET ratios of 0:1, 5:1, 10:1, 20:1 and 40:1 and incubated for 24 hr. As target cells, prostate cancer antigen-expressing cells were used. One day before mixed with effector cells, the target cells were cultured at a density of 1×10⁴ cells/well in a 96-well plate and then used. A control not containing effector cells underwent fixation and staining and was used for calculating specific lysis activities. At 24-hr after mixing effector and target cells, the supernatant of cells was removed and treated for 1 hr with 100 μl of 10% formalin for fixation. Following the removal of formalin, cells were stained using 80 μl of 4% crystal violet for 30 min and washed three times with tap water, followed by drying on air. Cells were treated with 80% methanol for 10 min. Finally, the OD values were measured at 570 nm. The specific lysis activity was calculated according to (sample OD/control OD)×100.

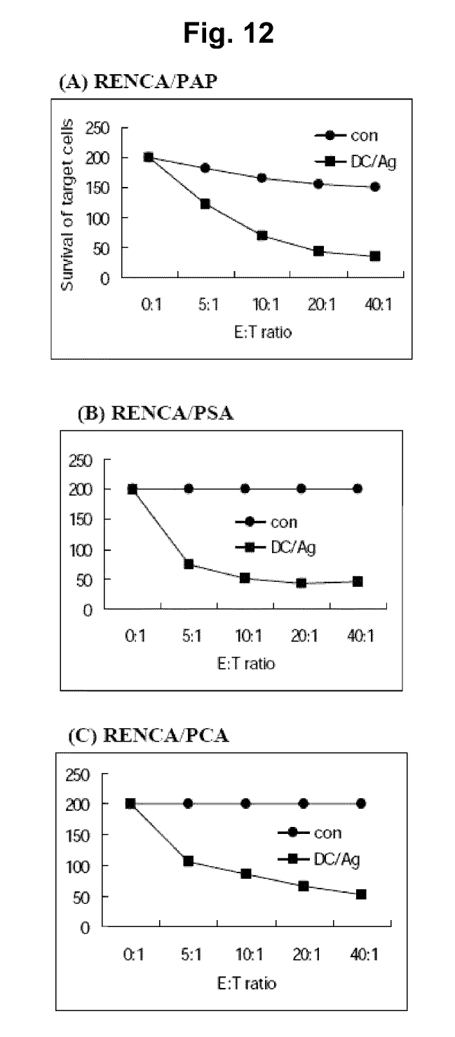

As represented in FIG. 12, CTLs were effectively induced in all three mouse models. These results demonstrate that the administration of dendritic cell-based vaccines pulsed with CTP-PAP, CTP-PSA or CTP-PCA induces effectively CTLs specific to human prostate cancer antigens, giving rise to the prevention and treatment efficacy to cancer. For experiments using cells expressing the PAP antigen, since mice themselves might have CTL clones, pulmonary metastasis was less than other cell lines and CTL activity was observed in control. For experiments using cells expressing the PSA or PCA antigen, only Ag-pulsed dendritic cells exhibited strong CTL activities. These results represent that CTLs having anti-cancer activity are effectively induced by dendritic cell-based vaccines. Interestingly, dendritic cells pulsed with the CTP-PCA antigen induce effectively the immune reactions against PCA-expressing cell lines. Tumor cells formed by PCA-expressing cancer cell lines show the increase in the proliferation due to the expression of PCA, so that anti-cancer efficacy of dendritic cells pulsed with the CTP-PCA antigen was measured to be relatively low. In other words, it would be recognized that the anti-cancer efficacy of dendritic cells may reflect both the anti-cancer immune responses and the proliferation of tumor cells.

As described previously, the present invention provides methods for analyzing the prevention and treatment efficacy of dendritic cells as immunotherapeutics for prostate cancer by use of animal models. For executing the prevention or treatment of prostate cancer using dendritic cells in a clinical scale, it is prerequisite to verity the efficacy and safety of dendritic cells in animal models. The present invention allows for animal model-based evaluation of dendritic cells as immunotherapeutics. Dendritic cell-based vaccines (DC vaccines) selected by the present invention become promising candidates as immunotherapeutics for prostate cancer.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for analyzing the prevention and treatment efficacy of a dendritic cell-derived immunotherapeutic for prostate cancer using an animal model carrying tumors expressing a human prostate cancer-specific antigen, which comprises the steps of:
(a) (a′) administering to a normal animal other than human dendritic cells to be analyzed, or (a″) administering to a normal animal other than human a cancer cell line expressing the human prostate cancer-specific antigen to induce cancer in the normal animal;
(b) (b′) administering to the animal the cancer cell line expressing the human prostate cancer-specific antigen to induce cancer in the animal when the step (a′) is performed in the step (a), or (b″) administering to the animal with cancer dendritic cells to be analyzed when the step (a″) is performed in the step (a); and
(c) determining the prevention and treatment efficacy of the dendritic cells as immunotherapeutics for prostate cancer by measuring the formation or growth of cancer cells originated from the cancer cell line in the animal.

2. The method according to claim 1, wherein the animal is rodents.

3. The method according to claim 3, wherein the rodent is a mouse (Mus musculus).

4. The method according to 1, wherein the human prostate cancer-specific antigen is PCA (prostate cancer antigen), PSCA (prostate stem cell antigen), PSA (prostate-specific antigen), PAP (prostate acid phosphatase) or PSMA (prostate-specific membrane antigen).

5. The method according to claim 4, wherein the human prostate cancer-specific antigen is PAP.

6. The method according to claim 1, wherein the cancer cell line is derived from a mouse (Mus musculus).

7. The method according to claim 6, wherein the cancer cell line is syngeneic to the animal.

8. The method according to claim 1, wherein the administration of dendritic cells or the cancer cell line in the step (a) is carried out by subcutaneous injection.

9. The method according to claim 1, wherein the administration of dendritic cells or the cancer cell line in the step (b) is carried out by subcutaneous injection.

10. The method according to claim 1, wherein the cancer cell line expressing the human prostate cancer-specific antigen is not a prostate cancer cell-derived one.

11. A mouse-derived renal cancer cell line (RENCA) expressing a human prostate cancer-specific antigen, characterized in that the human prostate cancer-specific antigen is PCA (prostate cancer antigen), PSCA (prostate stem cell antigen), PSA (prostate-specific antigen), PAP (prostate acid phosphatase) or PSMA (prostate-specific membrane antigen) and the renal cancer cell line is not a prostate cancer cell-derived one.

12. The mouse-derived renal cancer cell line (RENCA) according to claim 11, wherein the cancer cell line is transformed with a vector containing a nucleotide sequence encoding an amino acid sequence spanning amino acids 30-204 of SEQ ID NO:1, an amino acid sequence of SEQ ID NO:3, or an amino acid sequence of SEQ ID NO:4.

13. The mouse-derived renal cancer cell line (RENCA) according to claim 11, wherein the cancer cell line is transformed with a vector containing a nucleotide sequence of nucleotides 88-612 of SEQ ID NO:6, a nucleotide sequence of SEQ ID NO:8, or a nucleotide sequence of SEQ ID NO:9.

14. The mouse-derived renal cancer cell line (RENCA) according to claim 13, wherein the cancer cell line is transformed with pcDNA3.1(+)-Tag/PAP, pcDNA3.1(+)-Tag/PSA or pcDNA3.1(+)-Tag/PCA.

15. The mouse-derived renal cancer cell line (RENCA) according to claim 11, wherein the cancer cell line is RENCA/PAP (KCTC 10808BP) expressing the PAP antigen.

16. A mouse prostate cancer model, characterized in that the mouse model has a cancer formed by inoculating the renal cancer cell line of claim 11 expressing the human prostate cancer-specific antigen, and the metastasis or growth of the cancer formed in the mouse model is inhibited by the treatment of dendritic cells pulsed with the human prostate cancer-specific antigen.

17. The mouse prostate cancer model according to claim 16, wherein the renal cancer cell line is syngeneic to the mouse.

18. A mouse prostate cancer model is used for performing the method claim 1, the mouse model having a cancer formed by inoculating a mouse-derived renal cancer cell line (RENCA) expressing a human prostate cancer-specific antigen,
wherein the human prostate cancer-specific antigen is PCA (prostate cancer antigen), PSCA (prostate stem cell antigen), PSA (prostate-specific antigen), PAP (prostate acid phosphatase) or PSMA (prostate-specific membrane antigen) and renal caner cell line is not prostate caner-derived,
wherein the metastasis or growth of the cancer formed in the mouse model is inhibited by the treatment of dendritic cells pulsed with the human prostate cancer-specific antigen.


SEQUENCE LISTING (10 sequences)

SEQ ID NO: 1 - protein, 204 aa, Homo sapiens
MGAPLAVALGALHYLALFLQLGGATRPAGHAPWDNHVSGHALFTETPHDMTARTGEDVEMACSFRGSGSPSYSLEIQWWYVRSHRDWTDKQAWASNQLKASQQEDAGKEATKISVVKVVGSNISHKLRLSRVKPTDEGTYECRVIDFSDGKARHHKVKAYLRVQPGENSVLHLPEAPPAAPAPPPPKPGKELRKRSVDQEACSL

SEQ ID NO: 2 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 3 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 4 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 5 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 6 - DNA, 615 bp, Homo sapiens
atgggggccccgctcgccgtagcgctgggcgccctccactacctggcacttttcctgcaactcggcggcgccacgcggcccgccggccacgcgccctgggacaaccacgtctccggccacgccctgttcacagagacaccccatgacatgacagcacggacgggcgaggacgtggagatggcctgctccttccgcggcagcggctccccctcctactcgctggagatccagtggtggtatgtacggagccaccgggactggaccgacaagcaggcgtgggcctcgaaccagctaaaagcatctcagcaggaagacgcagggaaggaggcaaccaaaataagtgtggtcaaggtggtgggcagcaacatctcccacaagctgcgcctgtcccgggtgaagcccacggacgaaggcacctacgagtgccgcgtcatcgacttcagcgacggcaaggcccggcaccacaaggtcaaggcctacctgcgggtgcagccaggggagaactccgtcctgcatctgcccgaagcccctcccgccgcgcccgccccgccgccccccaagccaggcaaggagctgaggaagcgctcggtggaccaggaggcctgcagcctctag

SEQ ID NO: 7 - DNA, 372 bp, Homo sapiens
atgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgccgccatccttgcgctgctccctgcactcggcctgctgctctggggacccggccagctatag

SEQ ID NO: 8 - DNA, 1161 bp, Homo sapiens
atgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaacttatcatgtattctgcgcatgacactactgtgagtggcctacagatggcgctagatgtttacaacggactccttcctccctatgcttcttgccacttgacggaattgtactttgagaagggggagtactttgtggagatgtactaccggaatgagacgcagcacgagccgtatcccctcatgctacctggctgcagccccagctgtcctctggagaggtttgctgagctggttggccctgtgatccctcaagactggtccacggagtgtatgaccacaaacagccatcaaggtactgaggacagtacagattag

SEQ ID NO: 9 - DNA, 786 bp, Homo sapiens
atgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctga

SEQ ID NO: 10 - DNA, 2253 bp, Homo sapiens
atgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgactcggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaa